(19) United States
(12) Patent Application Publication
(10) Pub. No.: US 2004/0072189 A1
(43) Pub. Date: Apr. 15, 2004

(54) Prognostic indicator

(76) Inventors: Arthur John Smith, Crosby (GB); Philip Spencer Rudland, Drive Myrtle Street (GB)

Correspondence Address: Kate H Murashige, Morrison & Foerster, Suite 500, 3811 Valley Center, San Diego, CA, 92130, US

(21) Appl. No.: 10/381,091

(22) PCT Filed: Sep. 10, 2001
(86) PCT No.: PCT/GB01/04017

(30) Foreign Application Priority Data
Sep. 20, 2000 (GB) 0023080.5

(51) Int. Cl. C12Q 1/68; G01N 33/574

(52) U.S. Cl. 435/6; 435/7.23

(57) ABSTRACT

A prognostic indicator for metastasis comprises an antibody directed against ostepontin.

The present invention relates to a prognostic indicator for metastasis, a vaccine against metastatic cancer, a method for treating metastases and a kit for diagnosing life threatening metastases.

Most cancers are thought to be due to alterations in specific genes caused either by mutation making their gene-product in someway more effective or by over expression of a normal gene giving an enhanced effect These oncogenes have largely been identified by introducing gene-length fragments of DNA from human cancers into a mouse fibroblast cell line, in culture, and selecting those cell lines that grow in an uncontrolled manner in liquid or semi-solid medium. The oncogenes themselves have been isolated by cloning the human DNA fragments away from the mouse DNA by standard recombinatorial techniques. Alternatively mutations can arise in genes that suppress the activity of oncogenes such as, for example, P53 or Rb, or which suppress the levels of their product such as, for example NM-23. These are referred to as tumor suppressor genes. In the commonly occurring cancers it is believed that between 5 and 7 such changes in oncogenes or tumor suppressor genes are required to produce a full-blown cancer.

The major forms of cancer, including breast cancer, lung cancer and colonic cancer, cannot be cured effectively because, although the current therapies may be effective against the primary tumors, they are largely ineffective against the disseminating or metastasizing cells, which ultimately kill the patient. Despite the enormous effort in cancer research very little is known at the molecular level about the most important like-threatening process, that of metastasis. Most of the oncogenes and suppressor genes that have been discovered have been found from their ability to promote uncontrolled growth of the mouse fibroblast cell line. The major problem in this field is that determining cell growth does not a give a measure of the process of metastasis. In fact, although uncontrolled growth is an important aspect of the initial events in the development of a cancer, the rate of growth of distant metastasis can be remarkably slow. Hence the process of metastasis is largely independent of processes involving cell grow*, except in its final phases. Therefore, it is unlikely that oncogenes and tumor suppressor genes will have much involvement in the process of metastasis and be useful diagnostic or therapeutic targets for control and elimination of metastatic disease.

A protein which has been implicated in the formation of metastasis in cancers is osteopontin (Oates, A. J. et al 1997 Invasion and Metastasis 17, 1-15). Osteopontin (OPN) is a secreted, integrin binding, calcium binding, negatively charged, glycosylated phosphoprotein of approximately 44 to 60 KDa molecular mass that has been implicated in both normal and pathological processes. OPN is found in all body fluids and in the extra cellular matrix of mineralized tissues, and is one of the more abundant members of the non-collagenous proteins in bone. Typically, it is found in bone, kidney, blood vessels, the inner ear, epithelial cells of the gall bladder, gastrointestinal tract, bronchi, mammary gland, urinary and reproductive tracts and salivary and sweat ducts, tissues subject to ntinuous renewal in addition to activated T lymphocytes. OPN has been shown to be expressed at high levels in malignant cells and in the blood of patients with metastatic disease, and consequently a role for OPN in malignancy has been postulated (Singer D. R. et al. Secreted phosphoproteins associated with neoplastic transformation, Cancer Res 48: 5770 to 5774, 1988). There are also a number of studies to show that blood OPN levels in breast cancer are markedly elevated by metastasis, with higher OPN levels corresponding to deceased survival rate (Singhal, H Clinic Cancer Res 3: 605-611, 1997; Bellahcene, A and Casranovo V Am J. Pathol 146:95-100, 1995).

The sequence of human OPN precursor has been elucidated, the translation of which is as follows (SEQ ID No. 1):

MRIAVI CFCLLGITCA IPVKQADSGS
SEEKQLYNKY PDAVATWLNP DPSQKQNLLA PQNAVSSEET
NDFKQETLPS KSNESHDHMD DMDDEDDDDH VDSQDSIDSN
DSDDVDDTDD SLQSDESHHS DESDELVTDF PTDLPATEVF
TPVVPTVDTY DGRGDSVVYG LRSKSKKFRR PDIQYPDATD
EDITSHMESE ELNGAYKAI PVAQDLNAPSD WDSRGKDSYE
TSQLDDQSAE THSHKQSRLY KRKANDESNE HSDVIDSQEL
SKVSREFHSH EFHSHEDMLV VDPKSKEEDK HLKFRISHEL
DSASSEVN

(Crosby, A. H. et al. Genomic organization of the human osteopontin gene; exclusion of the locus from a causative role in the pathogenesis of dentinogenesis imperfecta type II. Genomics 27(1), 155-160, 1995).

Osteopontin has also been shown previously as a prognostic indicator both for gastric (Ue, T et al Int J Cancer 79; 127-132, 1998) and breast cancer (Tuck, A B et al Int J Cancer 79; 502-508, 1998) but the differences in prognosis were far from absolute.

Despite such a large body of work relating to the presence of OPN in cancerous cells, it has not been possible to elucidate a role for OPN in cancer generally or metastasis in particular. It is an object of the invention to determine a practical benefit for patients in connection with the known presence of OPN in cancerous cells.

In accordance with the first aspect of the present invention there is provided a prognostic indicator for metastases comprising an antibody directed against osteopontin.

The applicant has found surprisingly that the spread of life-threatening metastasis is absent in individuals with breast cancer in which osteopontin is not expressed.

OPN expression may thus be causative in the process of metastasis. Thus, a means for alleviating or curing life threatening cancer by preventing expression of OPN may be possible by means of the invention.

The antibody useful in the present invention may be employed histologically for in situ detection of osteopontin gene products or conserved variants or peptide fragments thereof In situ detection may be accomplished by removing a histological specimen from a patient, then applying thereto an antibody of the present invention directed against osteopontin which may subsequently be visualized using a second labeled antibody. Through a use of such a procedure, it is possible to determine not only the presence of the osteopontin gene product, or conserved variants or peptide fragments, but also its distribution in the examined tissue. Using the present invention, those of ordinary skill will readily perceive that any other wide variety of histological methods, such as staining procedures, can be modified in order to achieve such in situ detection. Preferably only epithelial cells of the carcinoma are examined; staining due to macrophages, host stroma, etc. is ignored.

For example, antibodies, or fragments of antibodies, such as those described hereabove may be used to detect the presence of osteopontin or conserved variants or peptide fragments thereof or labelled cDNA antisense probes may be used to detect the mRNA. This can be accomplished, for example, by immunofluorescent techniques employing a fluorescently labeled antibody coupled with light microscopic, flow cytometric, or fluorometric detection.

Assays for osteopontin gene products or conserved variants or peptide fragments thereof will typically comprise incubating a sample, such as a tissue extract, freshly harvested cells or lysates of cells which have been incubated in cell culture, in the presence of a detectably labeled antibody capable of identifying osteopontin gene products or conserved variants or peptide fragments thereof, and detecting the bound antibody by any of a number of techniques well known in the art.

The biological sample may be brought into contact with and immobilized onto a solid support or carrier such as nitro cellulose, or other solid support which is capable of immobilizing cells, cell particles or soluble protein. The support may then be washed followed by treatment with detectably labeled osteopontin specific antibody or fragments of antibodies. The solid support may then be washed with a buffer a second time to remove unbound antibody. The amount of bound label on solid support may then be detected by conventional means.

In accordance with a second aspect of the present invention there is provided a vaccine comprising an antigenic peptide that will generate an antibody directed against osteopontin.

The peptide may be derived from at least 10 consecutive amino acids of osteopontin. Preferably the peptide is derived from 14 to 20 consecutive amino acids of osteopontin. More preferably the peptide is derived from the amino acids from the amino terminus of osteopontin, since the amino terminus is extracellularly exposed. More preferably still the peptide is derived from amino acids from the region 28 to 48 (SEQ ID No. 2) of the human OPN precursor sequence described hereinabove: EEKQLYNKY PDAVATWLNP DP.

Even more preferably still, the peptide is derived from amino acids from the region 32 to 45 (SEQ. ID No. 3) of the human OPN precursor sequence described hereinabove: QLYNKYPDAVATWL.

The peptide may comprise an amino acid sequence which is at least 70% homologous to SEQ ID No. 2, preferably the peptide comprises at least 80% homology with SEQ ID No. 2 and more preferably the peptide comprises at least 90% homology with SEQ ID No. 2. Still more preferably the peptide comprises at least 70% sequence homology with SEQ ID No. 3, even more preferably still, the peptide comprises at least 80% sequence at least homology with SEQ ID No. 3 and most preferably the peptide comprises at least 90% sequency homology with SEQ ID No. 3.

Preferably the vaccine further comprises adjuvant: presently, alum (aluminium hydroxide and/or aluminium phosphate) is the only adjuvant approved for general use in human vaccines. Other adjuvants, notably Freund's complete, have been used in animals and are more effective, but toxic side effects have so far precluded their use in humans. Aluminium salt adjuvants are typically used with protein adjuvants in two maimers, (a) as alum-precipitated vaccines and (b) as alum-adsorbed vaccines (Harlow, E & D. Lane, 1988, Antibodies: A Laboratory Manual Cold Spring Harbor Laboratory; Nicklas, W., 1992, Aluminium salts. Research in Immunology 143:489-493. Alum is typically commercially available as Al (OH)₃ (Al hydrogel-superfos of Denmark/Accurate Chemical and Scientic Co, Westbury, N.Y.).

In one embodiment of the second aspect of the present invention the antigenic peptide may be coupled to a carrier protein.

In accordance with a third aspect of the present invention there is provided a method for treating metastases comprising administering a compound that modulates the expression of osteopontin.

In one embodiment, the expression of osteopontin may be blocked.

The compound may be an antibody directed against osteopontin, it may provide an antisense molecule that blocks translation of the osteopontin mRNAs or it may provide a nucleic acid molecule that is complementary to the 5′ region of the osteopontin gene and blocks transcription.

The compound may also be any small molecule which modulates the expression. The compound may block the induction of expression of osteopontin either by blocking transcription or translation of osteopontin, or by preventing its induction by interacting with T cell factor TCF) 4 or the small molecule may interact with a CAAAG sequence on DNA to prevent its sequestering of TCF4 and hence prevent induction of osteopontin (El Tanani et al. Oncogene 20, 1793-97 (2001); El Tanani et al. Cancer Research 61, 5619-5629 (2001)). The compound may also prevent interaction of osteopantin with intergrin alpha nu beta 1, integrin alpha nu beta 3, alpha nu beta 5 or alpha 4 beta 1 (Liaw L et al. J Clin Invest 95, 713-724 (1991); Miyaichi et al J. Biol Chem 266, 20369-20374 (1991); Bayless et al. J Cell Science 111, 1165-1174 (1998)). Preferably, the small molecule has a molecular weight less than 2 kDa.

In accordance with the fourth aspect of the present invention there is provided a kit for diagnosing metastasis comprising a prognostic indicator as described hereinabove and one or more of a visual indicator.

In accordance with a fifth aspect of the present invention, there is provided the use of a prognostic indicator as claimed in any one of claims 1 to 8 for determining whether a subject is at risk of developing metastasis comprising contacting a subject sample with the prognostic indicator and detecting the formation of a complex between the prognostic indicator and subject sample.

In accordance with a further aspect of the present invention, there is provided a method for determining whether a subject is at risk of developing metastasis comprising contacting a subject sample with a prognostic indicator as claimed in any one of claims 1 to 8 and detecting the formation of a complex between the prognostic indicator and subject sample.

A method for detecting the presence of osteopontin will now be described, by way of example only, with reference to the following examples and Figures:

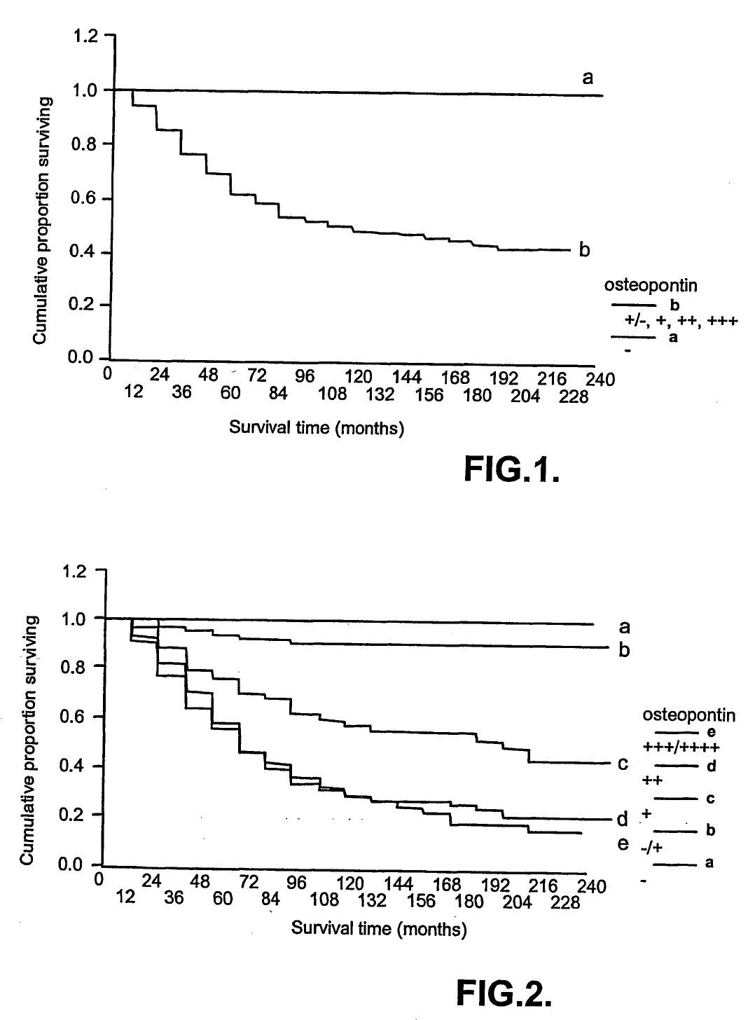

FIG. 1 Kaplan Meier survival curve for breast cancer patients in which primary tumor expressed different amounts of OPN, the positive staining groups are amalgamated.

FIG. 2 Kaplan Meier survival curve for breast cancer patients identified in FIG. 1 where groups are shown separately indicating a dose-response effect of expression of osteopontin.

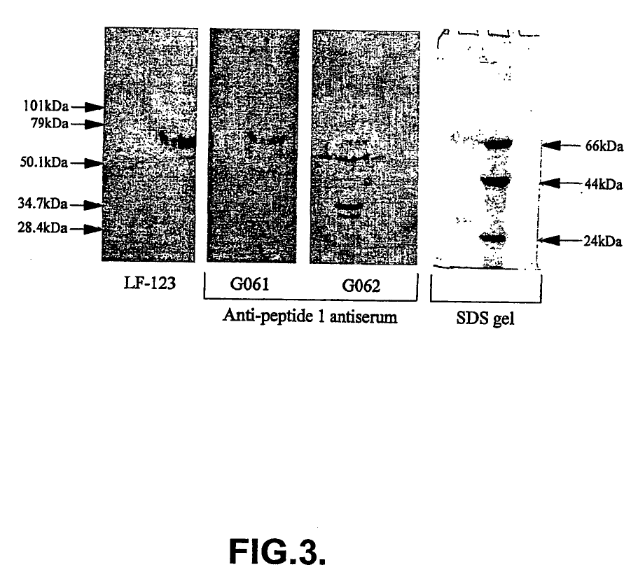

FIG. 3 Western blot illustrating the detection of peptide by antiserum raised against Cys+amino acids 32-45 of Rabbit osteopontin precursor SEQ ID No. 4 CQLYHKHPDALATWL

FIG. 1 and FIG. 2 illustrate Kaplan Meier survival curves where breast cancer tissues excised by surgery were collected from a group of 339 primary cancer patients, presenting with operable stage I and stage II forms of the disease, from within the Merseyside region, diagnosed between 1976 and 1982 at the Royal University Hospital (Winstanley et al, 1991 Br J Cancer 63: 447-450; 1993 Br J Cancer 67: 762-772). The age range was 29-92 (mean 57) at presentation Specimen tissues had been fixed routinely in neutral buffered formalin and preserved in paraffin blocks. Follow-up information was obtained and up-dated for patient survival to Aug. 31, 1995. The anti-osteopontin (alphaMBIII Bio (1) was from the Development Studies Hybridoma Bank, University of Iowa and is a monoclonal mouse antibody of IgG1 isotype and was used at a dilution of 1/30 in PBS containing 0.05% BSA. The second antibody was biotinylated sheep anti-mouse antibody (Amersham, Bucks) used at a dilution of 1/200 in PBS containing 0.5% BSA. The antibody was visualized using ABC complex (Dako, Bucks) and diaminobenzidine. Staining was assessed by two independent observers, recording the percentage of carcinoma cells with cytoplasmic staining for osteopontin from two sections of each specimen, 10 fields per section at 200× magnification. (Unstained cells were counterstained with Mayer's Haemalum). Staining levels of in situ carcinomas were ignored, as were staining of macrophages, lymphocyes, host stroma, spindle cells and blood vessels. Groups were defined as having <1% cells stained=ve, <5%=±, 5-25%=+, 25-50%=++, 50-75%=+++, 75-100%=++++. The groups contained 51, 66, 60, 95 and 67 carcinomas, respectively. Referring to FIGS. 1 and 2, differences between the groups are significant at the 5% level for all groups except—vs+/and+++vs+++.

The applicant has firther shown that MCF-7 cells (a human breast metastatic cell line) are recognised by the anti-osteopontin antibody described hereinabove when the cells are alive in culture, a clear indication that in vivo, the vaccine will work.

FIG. 3 illustrates a Western blot where Bovine osteopontin (3 μg) was electrophoresed in a 12% SDS gel and electroblotted onto a nitrocellulose membrane. The membrane was cut into three sections and each incubated overnight at 4° C. with a 1:1000 dilution of antiserum in Tris-buffered saline pH 7 containing 0.05% (v/v) TWEEN 20 (TBS-T). After washing in several changes of TBS-T, the membranes were incubated for 2 h at room temperature with a 1:1000 dilution of swine anti-rabbit immunoglobulins conjugated to horseradish peroxidase (Dako). Bound antibodies were visual using an ECL luminescent substrate kit (BioRad) and photographic film. By superimposing the developed film over the membrane, the positions of pre-stained proteins of known molecular weight present on the membrane could be indicated on the film. Anti-Peptide 1 antisera was raised against a 15 amino acid peptide of the rabbit osteopontin sequence. GO61 and GO62 refer to antiserum from two individual animals both inoculated with the peptide. LF123 was whole rabbit serum raised against recombinant human osteopontin.

Peptide CQLYHKHPDALATWL (Cys+amino acids 32-45 of osteopontin precursor) was synthesized commercially (Genosphere Biotechnologies, 2 Rue de Gravillieres, 75003, Paris, France) and coupled via cysteine to Keyhole Limpet Hemocyanin (KLH) (Lerner et al. 1981, PNAS 78,3404-3407). Two rabbits were injected with the construct together with adjuvant (4 injections at 3 week intervals), Freund's complete for first injection and Freund's incomplete for the others, and 2 weeks after the last injection were bled. The antiserum, at 1:10,000 dilution with phosphate buffered saline containing 1% bovine serum albumin and 0.01% sodium azide, detected peptide in ELISA and at 1:1,000 dilution detected bovine OPN by Western blot. One rabbit also recognised a smaller polypeptide at ˜35 kDa on the Western blot.

This demonstrates (i) that the peptide is antigenic and (ii) does not cause harm in the short term to the host.

1. A method for detecting the absence of metastatic cancer comprising the steps of:
i) contacting a subject sample comprising epithelial cells and/or lysates of epithelial cells from a primary tumour with a prognostic indicator comprising an antibody or a fragment thereof directed against osteopontin; and
ii) detecting the formation of any complex between the subject sample and prognostic indicator.

2. A method as claimed in claim 1 wherein the prognostic indicator for metastases comprises an antibody directed against an osteopontin gene product.

3. A method as claimed in claim 2 wherein the antibody is directed against a peptide derived from osteopontin.

4. A method as claimed in claim 3 wherein the peptide is derived from the amino acid terminus of osteopontin.

5. A method as claimed in claim 3 wherein the peptide comprises an amino acid sequence of at least 10 consecutive amino acids of SEQ ID No. 1.

6. A method as claimed in claim 5 wherein the peptide comprises an amino acid sequence of 14 to 20 consecutive amino acids of SEQ ID No. 1.

7. A method as claimed in any one of claims 5 or 6 wherein the peptide comprises an amino acid sequence which is at least 70% homologous to SEQ ID No. 2.

8. A method as claimed in claim 7, wherein the peptide comprises an amino acid sequence which is at least 90% homologous to SEQ ID No. 2.

9. A method as claimed in any previous claim wherein the method of diagnosis is for metastatic breast cancer.

10. A vaccine against metastatic cancer comprising an antigenic peptide derived from osteopontin.

11. A vaccine as claimed in claim 10, wherein the vaccine is against metastatic breast cancer.

12. A vaccine as claimed in any one of claims 10 or 11 wherein the antigenic peptide is derived from the amino terminus of osteopontin.

13. A vaccine as claimed in any one of claims 11 or 12 wherein the antigenic peptide comprises an amino acid sequence of at least 10 consecutive amino acids of SEQ ID No. 1.

14. A vaccine as claimed in claim 13 wherein the antigenic peptide comprises an amino acid sequence of 14 to 20 consecutive amino acids of SEQ ID No. 1.

15. A vaccine as claimed in any one of claims 13 or 14 wherein the antigenic peptide comprises an amino acid sequence which is at least 80% homologous to SEQ ID No. 2.

16. A vaccine as claimed in claim 15 wherein the antigenic peptide comprises an amino acid sequence which is at least 90% homologous to SEQ ID No. 2.

17. A vaccine as claimed in any one of claims 10 to 16 wherein the antigenic peptide is coupled to a carrier protein.

18. A vaccine as claimed in any one of claims 10 to 17 comprising an adjuvant.

19. A vaccine as claimed in claim 18 wherein the adjuvant is alum or Freund's Complete.

20. A method for treating metastatic cancer comprising the steps of:
i) diagnosing metastasis as claimed in any one of claims 1-8; and
ii) administering a compound that blocks the expression and/or function of osteopontin.

21. A method as claimed in claim 20 wherein the compound is an antibody directed against osteopontin.

22. A method as claimed in claim 20 wherein the compound provides an antisense molecule that blocks translation of the osteopontin mRNAs.

23. A method as claimed in claim 20 wherein the compound provides a nucleic acid molecule that is complementary to the 5′ region of the osteopontin gene and blocks transcription.

24. A method as claimed in claim 20 wherein the compound is a small molecule.

25. A method as claimed in claim 24 wherein the compound has a molecular weight which is 2 KDa or less.

26. A kit for diagnosing metastases as claimed in any one of claims 1-9 comprising a prognostic indicator and one or more of a visual indicator.

27. A method of diagnosis for metastatic cancer comprising the steps of:
i) contacting a subject sample comprising epithelial cells and/or lysates of epithelial cells from a primary tumour with a prognostic indicator comprising an antibody or a fragment thereof directed against osteopontin; and
ii) detecting the formation of any complex between the subject sample and prognostic indicator.


SEQUENCE LISTING (5 sequences)

SEQ ID NO: 1 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 2 - protein, 21 aa, Homo sapiens
EEKQLYNKYPDAVATWLNPDP

SEQ ID NO: 3 - protein, 14 aa, Homo sapiens
QLYNKYPDAVATWL

SEQ ID NO: 4 - protein, 15 aa, Rabbit
CQLYHKHPDALATWL

SEQ ID NO: 5 - protein, 5 aa, Homo sapiens
CAAAG